(19) United States
(12) Plant Patent Application Publication
(10) Pub. No.: US 2026/0305504 P1
(43) Pub. Date: Oct. 1, 2026

(54) MANGO TREE NAMED ‘BLYDE LATE’

(50) ;

(71) Applicant: Blydevallei International (Pty) Ltd, Hoedspruit (ZA)

(72) Inventor: Pieter Scholtz, Hoedspruit (ZA)

(21) Appl. No.: 19/555,351

(22) Filed: Mar. 3, 2026

Related U.S. Application Data
(60) Provisional application No. 63/779,675, filed on Mar. 28, 2025.

(51) Int. Cl. A01H 6/00; A01H 5/08

(52) U.S. Cl. CPC A01H 6/00; A01H 5/08
USPC PLT/159

(57) ABSTRACT

The new mango tree variety ‘Blyde Late’ is characterized by having large-size, elliptic, orange and purple colored fruit with a long shelf life and a very late harvest.

### CROSS-REFERENCE TO RELATED APPLICATIONS

This application claims the benefit of U.S. Provisional Application No. 63/779,675, filed on Mar. 28, 2025, which is incorporated by reference herein in its entirety.

Latin name of the genus and species claimed: Mangifera indica.

Variety denomination: ‘Blyde Late’.

### BACKGROUND AND SUMMARY OF THE INVENTION

This invention relates to the discovery and asexual propagation of a new and distinct variety of mango, Mangifera indica ‘Blyde Late’. The variety ‘Blyde Late’ was originated by open pollination and was first selected by Pieter Scholtz in 2017. The new variety was first evaluated by Pieter Scholtz in Hoedspruit, South Africa.

The seed parent is Heidi (unpatented) and the pollen parent is unknown. The parent varieties were first crossed in 2015. The new mango variety ‘Blyde Late’ was first asexually propagated by Pieter Scholtz in Hoedspruit, South Africa in 2018 by grafting.

The new variety ‘Blyde Late’ is characterized by having large-size, elliptic, orange and purple colored fruit with a long shelf life and very late harvest.

The new variety ‘Blyde Late’ differs from its seed parent Heidi (unpatented) in that the new variety has a light-orange-colored flesh, compared to an orange-colored flesh for ‘Heidi’. Additionally, the new variety ‘Blyde Late’ has a fruit weight of about 560 g, compared to 450 g for ‘Heidi’. Furthermore, the new variety ‘Blyde Late’ has a ripening date of May 1^{st}, compared to March 1^{st} for ‘Heidi’.

The new variety ‘Blyde Late’ differs from ‘Keitt’ (unpatented) in that the new variety has light-orange-colored fresh with an orange and purple-colored rind, compared to yellow-colored flesh with a yellow and orange-colored rind for ‘Keitt’. Additionally, the new variety ‘Blyde Late’ has a elliptic-shaped fruit with a weight of about 560 g, compared to a medium-elliptic shaped fruit with a weight of about 600 g for ‘Keitt’. Further, the new variety ‘Blyde Late’ differs from ‘Keitt’ in that the new variety has a ripening date of May 1st, compared to March 1st for ‘Keitt’.

The new variety ‘Blyde Late’ differs from ‘Brooks’ (unpatented) in that the new variety has ripening date of May 1st, compared to March 15^{th} for ‘Brooks’. Additionally, the new variety ‘Blyde Late’ has light-orange-colored flesh and an orange and purple-colored rind, compared to yellow-colored flesh and a yellow-green-colored rind for ‘Brooks’.

The new variety ‘Blyde Late’ has been shown to maintain its distinguishing characteristics through successive asexual propagations by, for example, grafting.

### BRIEF DESCRIPTION OF THE DRAWING

The accompanying color photographic illustrations were taken shortly after the fruit was picked from four-year-old plants and the colors are as nearly true as is reasonably possible in a color representation of this type.

FIGS. 1-3 show the fruit of the new variety ‘Blyde Late’.

FIG. 4. Show the fruit of the new variety ‘Blyde Late’ after storage at 12° C. from Apr. 24, 2024 to May 24, 2024.

FIG. 5 shows a cross-section of the fruit of the new variety ‘Blyde Late’, illustrating the flesh color and seed.

FIG. 6 shows the length of the new variety ‘Blyde Late’.

FIG. 7 shows the width of the new variety ‘Blyde Late’.

FIGS. 8-10 show the seed of the new variety ‘Blyde Late’.

FIG. 11 shows the leaves of the new variety ‘Blyde Late’.

FIG. 12 shows close-up views of a tree branch of the new variety ‘Blyde Late’.

FIGS. 13-15 show the tree of the new variety ‘Blyde Late’.

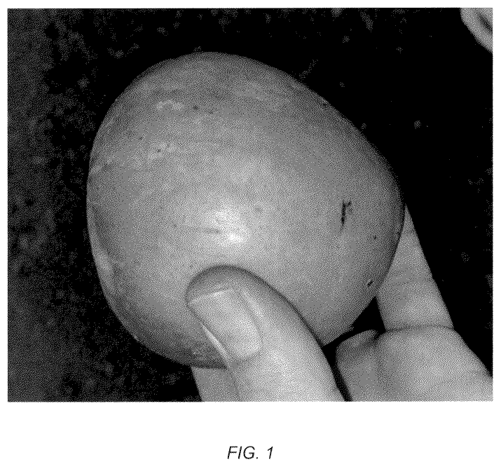

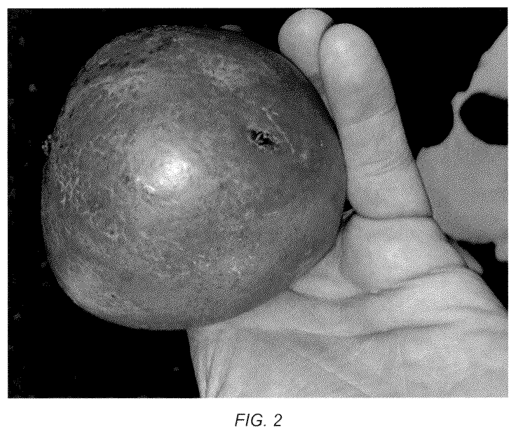

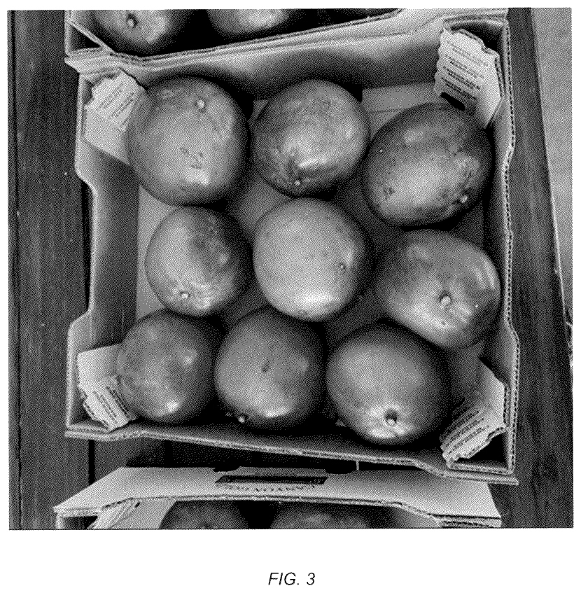

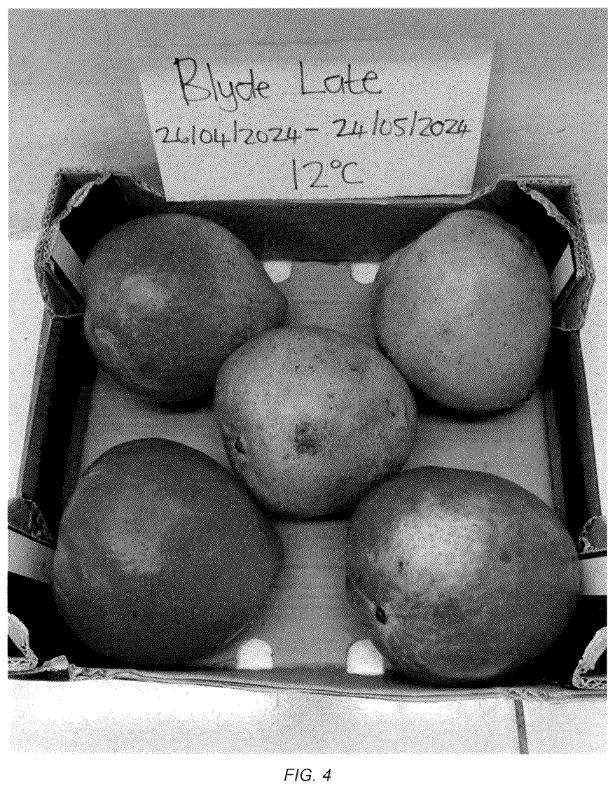

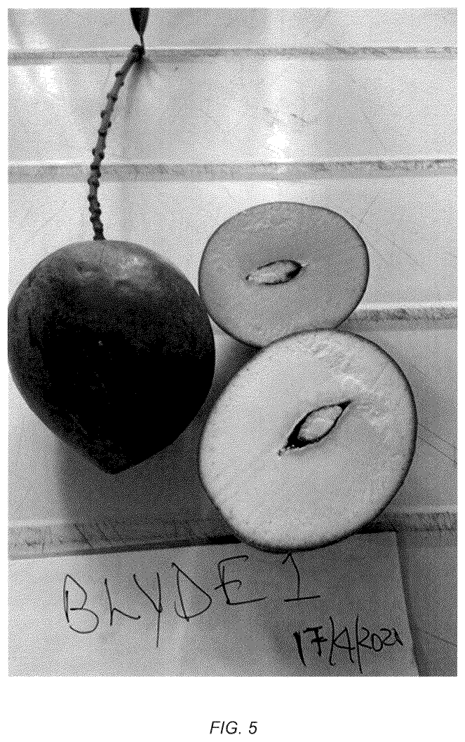

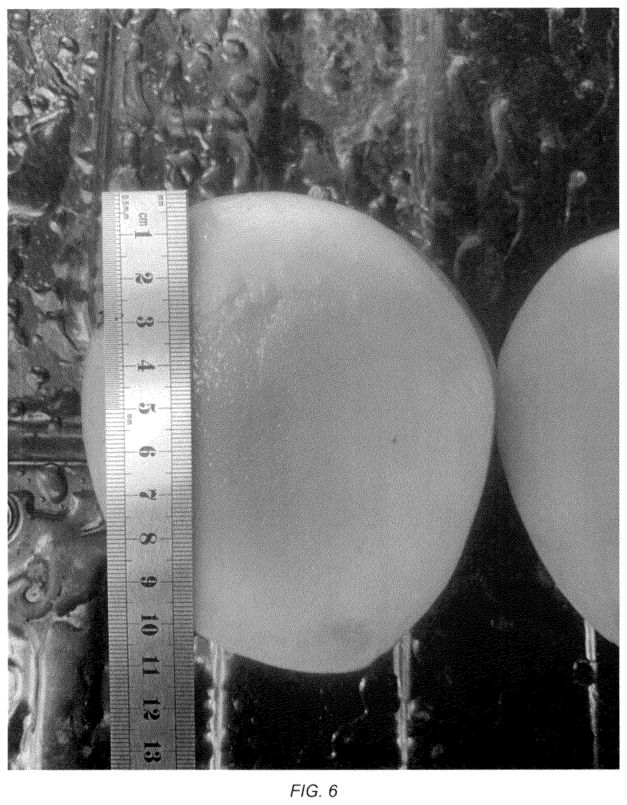

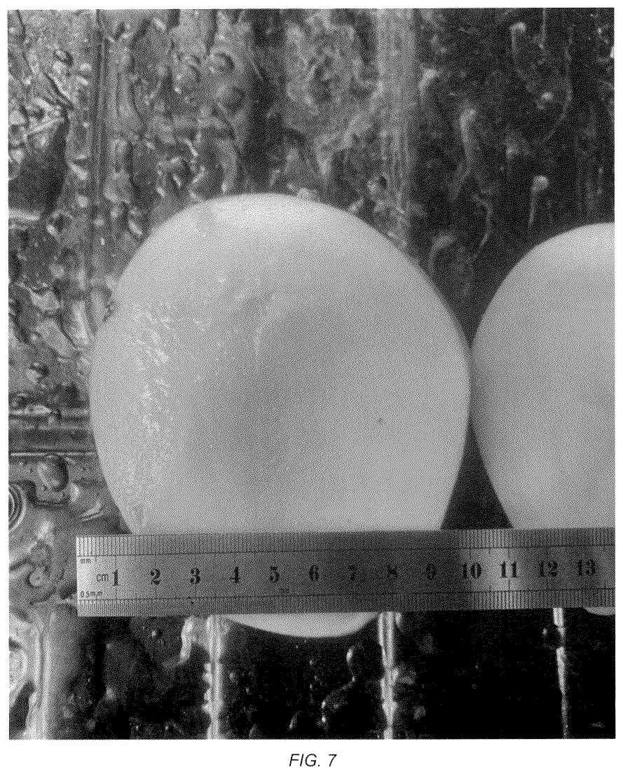

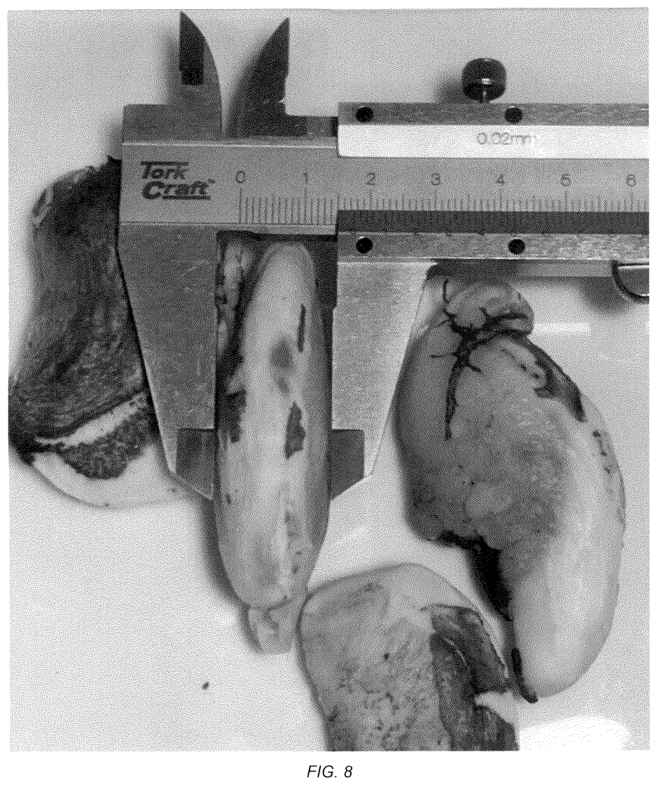

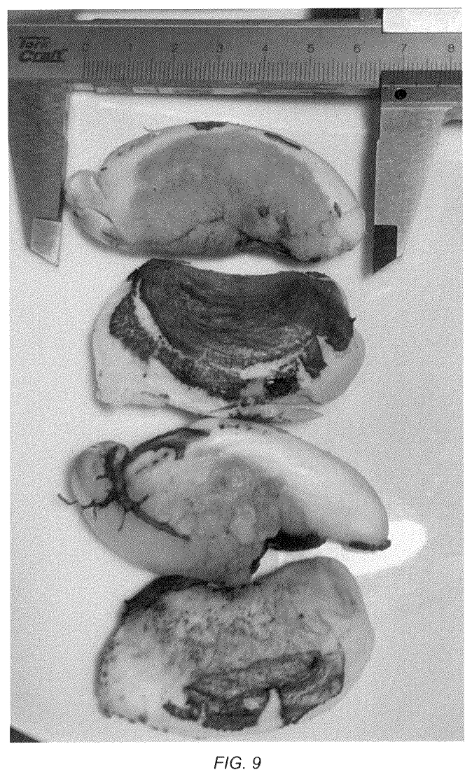

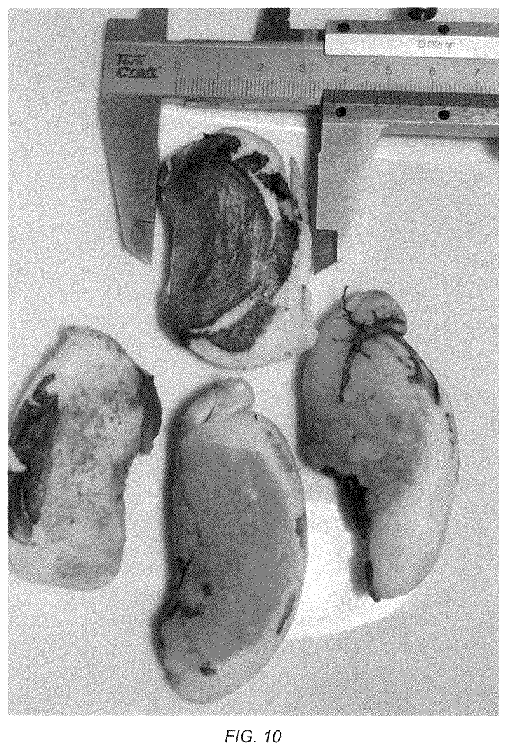

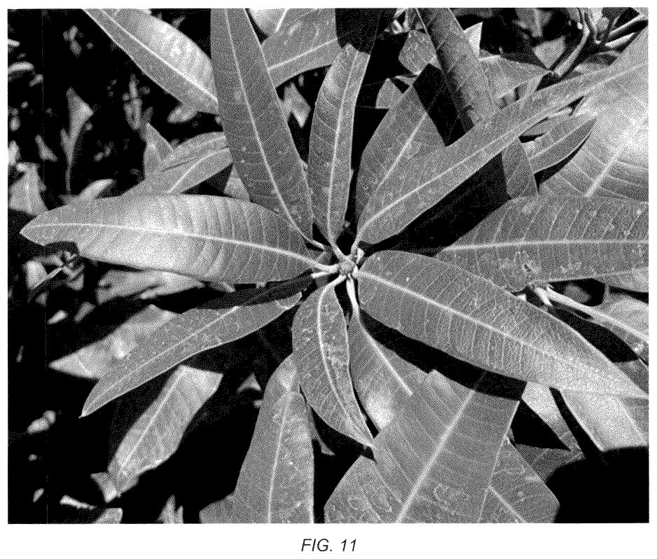

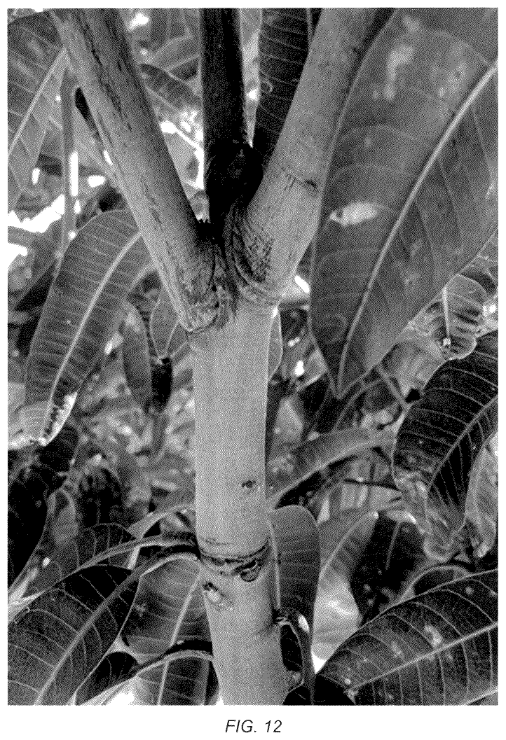

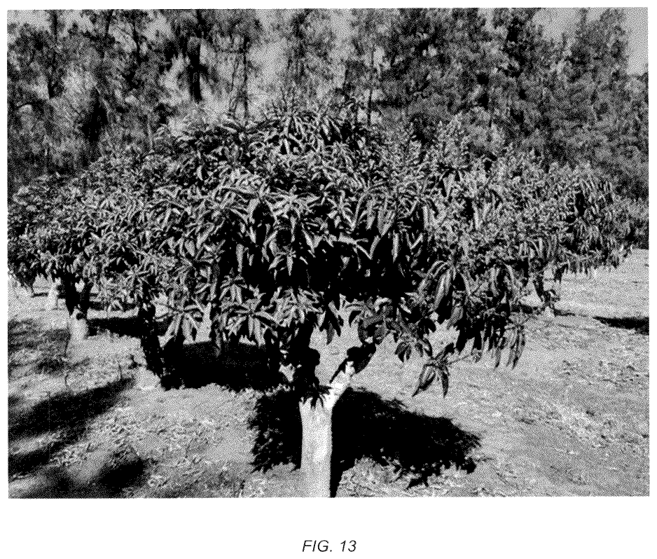

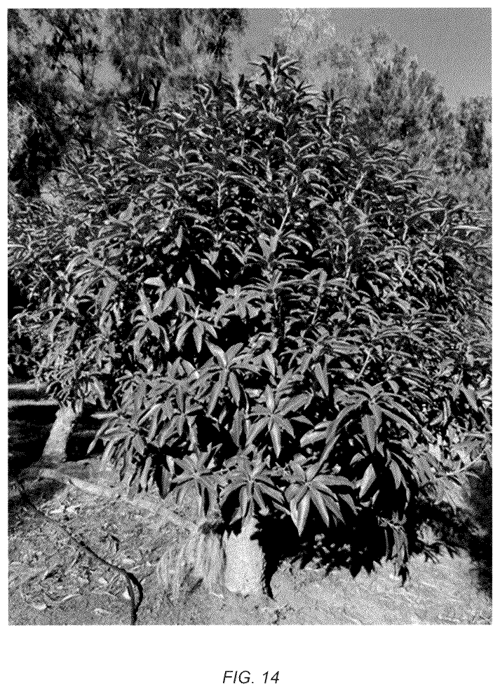

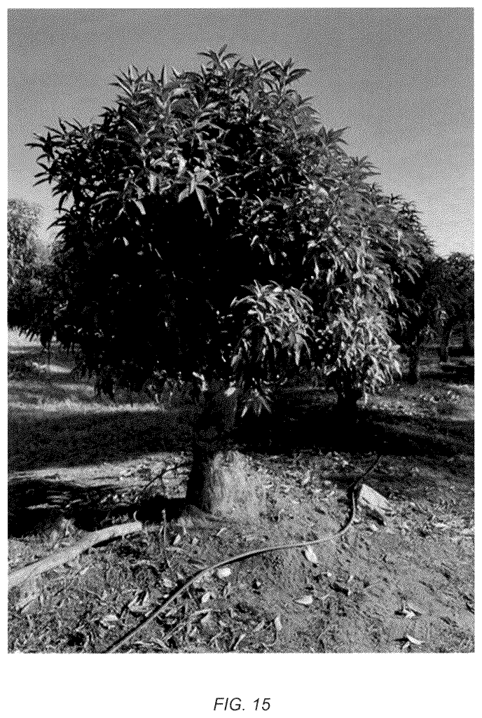

### DETAILED DESCRIPTION OF THE PREFERRED EMBODIMENT

Throughout this specification, color names beginning with a lower-case letter signify that the name of that color, as used in common speech, is aptly descriptive. Color names beginning with a capital letter designate values based upon The R.H.S. Colour Chart, sixth edition, published 2019.

The descriptive matter which follows pertains to two-year-old ‘Blyde Late’ trees grown in Hoedspruit, South Africa, and is believed to apply to plants of the variety grown under similar conditions of soil and climate elsewhere.
Tree:
General:
   Growth habit.—Spreading.
   Height.—Approximately 4 m.
   Average spread.—Approximately 3.5 m.
   Time of beginning of flowering.—Medium, about August 19^{th}.
   Flower duration.—About 52 days.
   Secondary off-season flowering.—Absent.
   Time of beginning of flowering (years).—2.
   Time of fruit maturity.—About May 1^{st}.
   Fruit bearing intensity.—Medium.
   Storage quality.—Good.
   Shelf life.—Good.
   Resistance and susceptibility to insects and pests.—Good.
   Plant hardiness.—Very hardy.
   Susceptibility to bruising.—Low.
   Susceptibility to wind.—Moderate.
   Heat tolerance.—Moderate.
   Brix.—About 16-20.
Trunk:
   Surface texture.—Smooth.
   Color.—About Medium Greyed-Orange N170.
Foliage:
Young leaves:
   Intensity of anthocyanin coloration.—Medium.
Leaf blade:
   Length.—Approximately 15.7 cm.
   Width.—Approximately 3.42 cm.
   Ratio length/width.—About 4.59:1.
   Shape.—Ovate.
   Color.—Upper surface: About Dark Green 135A. Lower surface: About Strong Green N134C.
   Shape of apex.—Acute.
   Shape of base.—Acute.
   Twisting.—Absent.
   Undulation of margin.—Absent or very weak.
   Spacing of secondary veins.—Medium.
   Leaf fragrance—crushed mature leaf.—Mild.
   Pubescence in upper and lower surfaces.—Absent.
Petiole:
   Attitude in relation to shoot.—Semi-erect.
   Length.—Short.
Flowers:
   Length of stamen in relation to pistil.—Longer.
   Number of stamens/staminodes.—About 5 (all fertile).
   Pistil color.—About Pale Greenish Yellow 2D.
   Anther color.—About Strong Red 53C.
Reproductive organs:
   Hemaphrodite flower.—Approximately 78%.
   Male flower.—Approximately 22%.
Inflorescence:
   Length.—Approximately 26.22 cm.
   Diameter.—Approximately 11.23 cm.
   Ratio length/diameter.—About 2.33:1.
   Number of primary branches.—Medium.
   Anthocyanin coloration of axis and branches.—Medium.
   Inflorescence color.—About pink.
   Axis growth habit.—Semi-erect.
   Shape.—Conical (narrowly Pyramidal).
   Peduncle color.—About Brilliant Yellow Green 150C.
   Peduncle diameter.—Approximately 1 mm.
Petal:
   Size.—Approximately 3 mm.
   Color.—About Vivid Yellow 14A.
Sepal:
   Size.—Approximately 2 mm.
   Color.—About Brilliant Greenish Yellow 151D.
Fruit:
   Overall size.—Large.
   Length.—Approximately 17.5 cm.
   Width.—Approximately 10.5 cm.
   Weight.—Approximately 560 g.
   Ratio length/width.—Approximately 1.66:1.
   Shape in cross section.—Broad elliptic.
   Fruit shape.—Elliptic.
   Predominant color of skin fully ripe.—About yellow and red.
   Color of skin, full sized fruit, at maturity.—About orange and purple.
   Speckling of skin.—Medium.
   Waxiness of skin.—Absent.
   Skin thickness.—Thick, approximately 0.65 mm.
   Density of lenticels.—Medium.
   Color contrast between lenticels and skin.—Medium.
   Size of lenticels.—Large.
   Roughness of surface (corkiness caused by lenticels).—Absent.
   Skin texture.—Smooth.
   Stalk cavity.—Absent or shallow.
   Fruit stalk insertion.—Oblique.
   Strength of fruit stalk attachment.—Medium.
   Presence of neck.—Absent.
   Shape of ventral shoulder.—Rounded outward.
   Shape of dorsal shoulder.—Rounded outward.
   Length of groove in ventral shoulder.—Absent or short.
   Depth of groove in ventral shoulder.—Absent or shallow.
   Bulging on ventral shoulder.—Absent.
   Presence of sinus.—Present.
   Depth of sinus.—Shallow.
   Point of stylar scar.—Medium.
   Bulging proximal of stylar scar.—Absent or weak.
   Diameter of stalk attachment.—Medium, approximately 0.6 cm.
   Stalk length.—Approximately 1.4 cm.
   Stalk diameter.—Approximately 0.5 cm.
   Adherence of skin to flesh.—Medium.
   Flesh Texture.—Medium.
   Main flesh color.—About Vivid Yellow 16A.
   Flesh firmness.—Medium.
   Juiciness.—Medium.
   Turpentine Flavor.—Mild.
   Amount of fiber attached to stone.—Medium.
   Amount of fiber attached to skin.—Low.
Stone:
   Color.—White.
   Length.—Approximately 7 cm.
   Width.—Approximately 4 cm.
   Thickness.—Approximately 2.5 cm.
   Venation.—Parallel.
   Relief of surface.—Grooved.
   Shape in lateral view.—Oblong.
Seed:
   Shape in lateral view.—Oblong.
   Length.—Approximately 6.5 cm.
   Width.—Approximately 3.5 cm.
   Embryony.—Monoembryonic.
   Seed space occupied inside stone.—About 51-75%.

What is claimed is:

1. A new and distinct variety of mango tree named ‘Blyde Late’ as illustrated and described herein.
